(19) United States
(12) Patent Application Publication
(10) Pub. No.: US 2017/0151082 A1
(43) Pub. Date: Jun. 1, 2017

(54) ORTHOPEDIC DEVICE FOR LIMITING THE MOVEMENT OF A JOINT ARRANGED BETWEEN A FIRST AND A SECOND BODY REGION

(71) Applicant: BETTERGUARDS TECHNOLOGY GmbH, Berlin (DE)

(72) Inventor: Vinzenz BICHLER, Berlin (DE)

(21) Appl. No.: 15/313,803

(22) PCT Filed: May 22, 2015
(86) PCT No.: PCT/EP2015/061450

§ 371 Date: Nov. 23, 2016

(30) Foreign Application Priority Data
May 23, 2014 (DE) 10 2014 107 335.5

(51) Int. Cl. A61F 5/01

(52) U.S. Cl. CPC A61F 5/0111; A61F 5/0127; A61F 5/013; A61F 2005/0165

(57) ABSTRACT

An orthopedic device for limiting the movement of a joint arranged between a first and a second body region. The orthopedic device comprises at least a receptacle that can be fastened to the first body region and a pull-out body that can be fastened to the second body region and can be moved in relation to the receptacle, wherein a passage cross-section filled with a dilatant fluid is provided perpendicularly to a pull-out direction between the receptacle and the pull-out body, wherein at least one passage-limiting element is provided for changing the passage cross-section.

### CROSS-REFERENCE TO RELATED APPLICATIONS

The present application is a National Stage application of PCT Application No. EP2015/061450 and is claiming priority of German Patent Application No. 10 2014 107 335.5, filed on May 23, 2014, the content of each of which is herein incorporated by reference.

### TECHNICAL FIELD

The present invention relates to an orthopedic device for limiting the movement of a joint disposed between a first and a second body portion, comprising at least a reception fixable to the first body portion and a pull-out body fixable to the second body portion and movable relatively to the reception, wherein perpendicular to a pull out direction between the reception and the pull-out body a passage cross section filled with shear thickening fluid is provided.

### TECHNOLOGICAL BACKGROUND

Damages in the area of joints involved in the cycle of movement belong to one of the most important accident focal points. In particular, emphasis is put on “accidents relating to tripping, slipping and falling” (TSF-accidents), which most frequently occur as accidents relating to commuting, working, and sports. Separately or in combination these accidents can lead to significant injuries. Especially, larger joints as the upper and lower ankle joint (articulatio talocuralis and articulatio talotarsalis), the wrist (articulatio manus) and the knee joint (articulatio genus) are mainly affected. Thereby, in this area a tendency to injury can also be registered in terms of sports medicine.

The TSF-accidents often lead to a violent strain of the support structure of the joint (joint capsule, ligaments, tendons, bones) beyond the physiological maximum possible angle. This strain is also known under the term distorsion or sprain. As in terms of the ankle joint and the wrist this often occurs in the direction of supination, in this case it is spoken of a so-called supination trauma. This is a synonym for an injury, wherein a supination of the foot that was made beyond the physiological room for maneuver together with a plantar flexion leads to a damage of the lateral bone structures and the lateral collateral ligaments apparatus. Initially, supination traumas are revealed in severe pain that mostly is linked to a swelling of the appendage and to the migration of a hematoma (in the case of ruptured ligaments). If such accompanying symptoms are not recognized and medical care is not sufficiently provided, remote damages such as a chronic instability of the joints or joint arthrosis may occur. The cause of injury for supination traumas often are high supination (inversion) speeds and exceeding a critical angle responsible for the injury, such as a twisted ankle. Thus, in the case of a twisted ankle, for example, a critical angular speed of more than 300/s can lead to an injury. Hereinafter, characteristic values below this critical angular speed for embodiments for the ankle joint are referred to as physiological movements. The occurring speeds result from forces or accelerations acting on the body portions and joints.

A very important factor for the positive healing process of a supination trauma is the stabilization of the joints of the patient for the prevention of injuries or during care before or after an operation. Thereby, the stabilization of the joints is normally achieved through tape strappings, orthoses for the assurance of function, orthoses of stabilization, bandages and/or stable shoes with a shaft with variable height and optional reinforcement elements.

In general and in this application, orthosis can be understood as a function assuring, body enclosing or body adjacent aid that in terms of its physical/mechanical performance constructively stabilizes, immobilizes, mobilizes, relieves, corrects, retains, fixes, redrills and/or replaces failed body functions.

In particular, dynamic orthoses for regulating the force between body portions relatively movable towards each other are used. Orthosis are known that have a movability depending on a shear thickening fluid.

For an adaptive limitation of movement shear thickening materials such as copolymer dispersions or fluids are used, which show a higher viscosity when high shear forces and an accompanying high shear rate occur. The higher the applied shear is, the more viscose or thicker is the behavior of the fluid. Accordingly, shear strengthening or shear thickening properties are attributed to shear thickening fluids.

Orthoses using the properties of such shear thickening fluids, for example, can have a reception and a pull-out body. The reception is filled with the shear thickening fluid. The pull-out body protrudes into the reception so that at least the area of the pull-out body overlapping with the reception is surrounded by the shear thickening fluid. The pull-out body can be attached to a first body portion and the reception can be attached to a second body portion which is relatively movable to the first body portion.

When a defined small force is applied, the pull-out body can be moved in the shear thickening fluid almost unrestrictedly. When a high force is applied, which results in a strong or abrupt tension on the pull-out body the critical shear rate in the shear thickening fluid is exceeded. Depending on the used fluid this results in an increase of the tensile stress by the factor 100 to the factor 1000. The increase of the tensile stress has a linking effect on the shear thickening fluid, whereby the pull-out body, on which a high force is applied, is held in the shear thickening fluid by means of the strong increase of the viscosity of the shear thickening fluid and, thus, undergoes no or only a reduced movement.

For example, U.S. Pat. No. 5,712,011 A shows a tug resistant unit wherein a first member has an inextensible section with a surface overlapping a surface of a second inextensible member. The two members are arranged in a way that they allow for a translational substantially parallel movement relative to one another. A shear-thickening composition providing a variable resistance against the translational movement of the second member relative to the first member is disposed between the members. The resistance results from the shear forces acting on the shear-thickening composition, which result from the relative movement of the inextensible members towards one another. The inextensible members and the shear-thickening composition may be enclosed within an enclosure.

Present devices enable either a strong protective effect and, thus, an immobilization, or a light protective effect that only protects insufficiently in a case of injury. Hence, for different load scenarios a change of the device or a manual default setting is necessary in order to adapt the properties of the device to the course of healing.

It is an object of the present invention to provide an improved orthopedic device.

### SUMMARY OF THE INVENTION

Starting from the known prior art it is an object of the present invention to provide an orthopedic device for limiting the movement of a joint disposed between a first and a second body portion which improves the use of the orthopedic device and enlarges the range of application.

This object is solved by means of a device with the features of claim 1. Further embodiments can be taken from the dependent claims.

Accordingly, an orthopedic device for limiting the movement of a joint disposed between a first and a second body portion is given, comprising at least one reception fixable to the first body portion and a pull-out body fixable to the second body portion and movable relative to the reception, wherein perpendicular to the pull-out direction between the reception and the pull-out body a passage cross-section filled with a shear thickening fluid is provided. According to the invention at least one passage limiting element for changing the passage cross-section is provided.

At least one passage limiting element may involve that exactly one passage limiting element is provided in the orthopedic device. Alternatively at least one passage limiting element may also involve that a plurality of passage limiting elements is provided in the orthopedic device, such as two, three, four, five, ten, 20, 40, 50, 64 or 100 passage limiting elements.

On a plane perpendicular to the pull-out direction seen over the length of the pull-out body the passage cross-section represents the smallest distance between the pull-out body and the reception. Thus, seen over the length of the pull-out body the passage cross-section forms the smallest passage for a shear thickening fluid between the pull-out body and the reception.

The at least one passage limiting element can be disposed at the pull-out body and/or at the reception. Alternatively, also several passage limiting elements can be disposed at the pull-out body and/or at the reception. The at least one passage limiting element is intended to change the passage cross-section. In a starting position of the at least one passage limiting element the maximum passage cross-section between the pull-out body and the reception is provided. Depending on the speed by means of which the pull-out body moves relatively to the reception, in particular by means of which the pull-out body is being pulled out of the reception, the at least one passage limiting element can take over a position different from the starting position, by means of which the passage cross-section between the pull-out body and the reception is being reduced.

In a maximum limiting position the greatest possible reduction of the passage cross-section is provided by the at least one passage limiting element.

Due to the fact that the passage cross-section can be changed by means of the at least one passage limiting element, the viscosity and the shear rate of the shear thickening fluid in the orthopedic device can be influenced. Hence, a reduction of the passage cross-section can lead to the shear thickening fluid having a significantly higher viscosity at the same shear rate. Furthermore, due to a reduced passage cross-section a jump in dilatancy, i.e. the start of shear thickening, can be achieved at significantly lower shear rates.

The position of the at least one limiting element and, thus, the size of the passage cross-section has a dependent relationship with the relative speed of the pull-out body over the reception. At low speeds which are uncritical for the joint to be protected with the orthopedic device, so-called physiological speeds, the at least passage limiting element remains in the starting position or deviates only slightly from the starting position, so that the passage cross-section between the pull-out body and the reception is only slightly reduced. Thus, slow relative movements of the pull-out body with respect to the reception can be enabled due to low viscosity of the shear thickening fluid.

With increasing relative speed, in particular pull-out speed, of the pull-out body with respect to the reception the passage cross-section between the pull-out body and the reception is increasingly reduced by means of the at least one passage limiting element. Due to the reduction of the passage cross-section between the pull-out body and the reception, the viscosity of the shear thickening fluid rises. Furthermore, the required shear rate for the start of the shear thickening is reduced.

With an increasing deflection of the at least one passage limiting element also the flow resistance caused by the at least one passage limiting element rises. Due to the increasing deflection a projected area increases in pull-out direction of the at least one passage limiting element which results in an enlarged pull-out resistance under an increased generation of turbulences and whirlings. The projected area is formed when the at least one passage limiting element is projected parallel to the pull-out direction onto a plane perpendicular to the pull-out direction. The generated turbulences and whirlings lead to an additional solidification in the shear thickening fluid depending on the relative speed. This shear thickening effect is called pseudo dilatant behavior.

Thus, it can be achieved that the relative movement of the pull-out body with respect to the reception is from limited to completely suppressed.

In a further embodiment the at least one passage limiting element is plate-shaped, preferably disc-shaped, more preferably flake-shaped, and/or rod-shaped, preferably bristle-shaped.

Thereby, in a position of the at least one passage limiting element different from the starting position, the passage cross-section can be reduced by means of the surface of the at least one passage limiting element. The bigger the area of the passage cross-section which is covered by the surface of the at least one passage limiting element, the smaller is the passage cross-section.

The at least one plate-shaped and/or rod-shaped passage limiting element for deflection from a starting position can be disposed parallel to the longitudinal direction of the pull-out body and of the reception in a starting position. Opposite the first end of the at least one passage limiting element which is firmly attached to the pull-out body or the reception, a second end of the at least one passage limiting element may be provided. The at least one passage limiting element has a first surface which in the starting position faces the pull-out body, and a second surface which in the starting position faces the reception. The second free end of the at least one plate-shaped and/or rod-shaped passage limiting element points towards the pull-out direction in the starting position.

Thus, the plate-shaped, preferably disc-shaped, more preferably flake-shaped, and/or rod-shaped, preferably bristle-shaped at least one passage limiting element for the deflection from a starting position can change the passage cross-section by means of a deflection from the starting position which is caused by the pull-out speed, so that the viscosity of the shear thickening fluid may be influenced subject to the pull-out speed. In particular, the at least one passage limiting element provides an increase of turbulences and whirlings in the shear thickening fluid.

In a further embodiment the at least one passage limiting element for deflection from a starting position may be flexible and on a first end firmly, preferably in a torque-proof manner, attached to the pull-out body or the reception.

When pulling out the pull-out body from the reception the shear thickening fluid flows around the at least one passage limiting element. Due to the flexible properties of the at least one passage limiting element and due to the effect of the flowing fluid the at least one passage limiting element can be bent from the starting position towards the maximum limiting position depending on the pull-out speed. Thereby, the passage cross-section which depends on the distance of the second end of the at least one passage limiting element from the reception and/or the pull-out body can be reduced.

The at least one flexible passage limiting element can change the passage cross-section by means of the deflection from the starting position which it undergoes subject to the pull-out speed, so that the viscosity of the shear thickening fluid can be influenced subject to the pull-out speed. In particular, the at least one passage limiting element provides an increase of turbulences and whirlings in the shear thickening fluid.

If the pull-out body is moved into the reception against the pull-out direction, the shear thickening fluid flowing around the at least one passage limiting element supports the return of the at least one passage limiting element to the starting position.

Furthermore, when the pull-out speed is reduced or omitted the at least one passage limiting element can be returned to the starting position due to its flexible properties.

In a further embodiment the at least one passage limiting element for deflection from a starting position may be attached to the pull-out body or the reception on a first end via a joint, preferably a hinge, more preferably a film hinge.

Thus, in a starting position the at least one passage limiting element can be folded so that it lies flat to the pull-out body and/or the reception, whereby the maximum passage cross-section between the reception and/or the pull-out body and the at least one passage limiting element, in particular, the second end of the at least one passage limiting element, is provided.

In the starting position the second end preferably points in the pull-out direction. If the pull-out body is pulled out of the reception, the at least one passage limiting element falls out due to an interaction with the flowing shear thickening fluid. Thereby, the second end of the at least second passage limiting element approximates the reception and/or the pull-out body, thus, reducing the passage cross-section.

The folding-out of the at least one passage limiting element is enabled by means of the joint which attaches the at least one passage limiting element on the first end to the pull-out body or the reception.

Furthermore, the at least one passage limiting element can be fold-out until a maximum limiting position. For example, the limiting position can be provided by means of a stop at the first end of the at least one passage limiting element, which touches the joint, the surface of the pull-out body, or the reception.

If the pull-out body is being pulled into the reception the shear thickening fluid flows around the at least one passage limiting element in a way that it is folded to its starting position. In particular, the shear thickening fluid presses on a second surface of the at least one passage limiting element and, thus, provides the return of the at least one passage limiting element to its starting position.

Additionally, the joint can have a return spring for returning the at least one passage limiting element to its starting position after the omission of the pull-out movement. The joint can be formed in the form of a hinge. The hinge on the first end of the at least one passage limiting element can be disposed on the pull-out body or the reception in order to enable folding-in and folding-out of the at least one passage limiting element.

Furthermore, the hinge can be formed in a way of a film hinge. Thereby, the at least one passage limiting element can be formed integrally with the pull-out body or the reception. The film hinge can be formed out of a thin walled connection which enables a limited rotary motion of the connected parts due to its flexibility. The one-piece design can consist of a multi-component injection molding, for example.

The connection of the at least one passage limiting element with the pull-out body or the reception via a joint, hinge, or film hinge enables that the at least one passage limiting element can be folded out and folded in. Thereby, it is possible to change the passage cross-section between the pull-out body or the reception and the at least one passage limiting element.

In a further embodiment the at least one passage limiting element may be disposed in an acute angle to the pull-out body in a starting position. The at least one passage limiting element can function as an anchor. Hence, a first end of the at least one passage limiting element is firmly attached or connected via a joint to the pull-out body. The second end of the at least one passage limiting element protrudes from the pull-out body like the tip of the fluke of an anchor. In the starting position the second end of the at least one passage limiting element substantially points to the pull-out direction.

If the pull-out body is being moved in the pull-out direction the shear thickening fluid flows onto a first surface which lies on the inside with respect to the pull-out body and exerts a force on that first surface. Depending on the attachment of the at least one passage limiting element on the pull-out body the at least one passage limiting element folds or bends towards the reception and thereby limits the passage cross-section between the reception and the second end of the at least one passage limiting element.

The passage limiting element may face a second passage limiting element on the other side of the pull-out body. Thereby, it can be ensured that the pull-out body is being pulled out of the reception in a parallel manner. Thus, moments of force which arise due to the leverage originating from the fluid forces acting on the passage limiting elements can be balanced so that the pull-out body is held in an orientation parallel to the reception and is not pushed to the inner surface of the reception. Thus, a constant distance between the passage limiting elements opposing each other and the reception can be provided.

In a further embodiment the pull-out body may be surrounded by at least one umbrella shaped passage limiting element. In this case, the pull-out body can be surrounded by eat least one continuous passage limiting element which circumferentially surrounds the pull-out body with respect to the pull-out direction.

In a starting position the at least one umbrella-shaped passage limiting element and the pull-out body form a closed shield. A first edge surrounding the pull-out body forms a torque-proof connection or joint connection with the at least one passage limiting element. A second free edge of the shield surrounding the pull-out body can move away from the pull-out body by means of pulling out the pull-out body and, thus, stretch the umbrella-like passage limiting element. Thereby, the passage cross-section between the reception and the second circumferential edge of the at least one umbrella-shaped passage limiting element can be reduced.

In the one-piece embodiment the at least one umbrella-shaped passage limiting element can comprise an elastic material, preferably rubber or natural rubber.

Alternatively, several single passage limiting elements can be disposed around the pull-out body and, thus, form a multi-piece shield around the pull-out body. In particular, the single passage limiting elements can be fan-like, i.e. they can be disposed around the pull-out body in a partially overlapping manner. Thereby, the requirements in terms of the flexibility of the passage limiting elements when reducing the passage cross-section is low. By means of a reduction of the overlapping areas among the passage limiting elements, which accompanies a movement of the second end towards the reception, an expansion of the circumference of the second circumferential edge which is formed by the second end of the passage limiting elements can be achieved.

Apart from the possibility of increasing the viscosity due to the reduction of the passage cross-section the umbrella-shaped embodiment of the at least one passage limiting element additionally may provide an increased flow resistance. Thus, when pulling out the pull-out body of the reception, the pull-out body may be slowed-down or stopped due to the umbrella-shaped design.

In a further embodiment the pull-out body and/or the reception may have at least one recess in which at least one passage limiting element is recessed in a starting position. Thus, the entire distance between the pull-out body and the reception perpendicular to the pull-out direction can be used as passage cross-section for the shear thickening fluid. Due to the force impact onto the orthopedic device, which is uncritical for the joint to be preserved, and the accompanying physiological speed, the pull-out body and the reception can be moved relative towards each other, wherein the passage cross-section is not influenced or reduced by the at least one passage limiting element.

Thereby, low component heights of the orthopedic device can be realized. No additional distance is required between the pull-out body and the reception for the provision of the at least one passage limiting element.

Furthermore, due to the recessed arrangement of the passage limiting elements in the starting position from the pull-out body or the reception no protrusions are provided due to the at least one passage limiting element, whereby less viscosity increasing whirlings and turbulences are created in the shear thickening fluid before an increase of the viscosity in the shear thickening fluid is desired. In particular, in terms of a physiological movement without increased effort a pre-mature limitation of movement of the orthopedic device can be countered. To the contrary, when the force impact is high, the flow around the first and second surfaces of the at least one passage limiting element can deviate the at least one passage limiting element from its starting position and, thus, can move the second end of the at least one passage limiting element towards the reception or the pull-out body, so that the passage cross-section between the second end of the at least one passage limiting element and the reception is reduced.

In order to facilitate a deflection of the at least one passage limiting element the at least one passage limiting element can be arranged slightly inclined with respect to the pull-out direction in the starting position.

Furthermore, at least one passage limiting element disposed in a recess of the pull-out body is opposed by a further passage limiting element disposed in a further recess of the pull-out body on the opposite side of the pull-out body perpendicular to the pull-out direction. Furthermore, for each passage limiting element the pull-out body has a further passage limiting element which is movable in a horizontal opposite direction with respect to the first passage limiting element. By reducing the passage cross-section by means of passage limiting elements opposing each other the forces originating from the passage limiting elements and acting on the pull-out body may be balanced. Though, an interaction of the passage limiting elements with the shear thickening fluid the pull-out body can be moved parallel to the reception without being pressed against the inner surface of the reception.

In a further embodiment the pull-out body may have at least one passage for providing an additional flow path for the shear thickening fluid, in which at least one passage limiting element is recessed in a starting position. Thereby, in a position differing from the starting position of the at least one passage limiting element the shear thickening fluid additionally can flow through the passage of the pull-out body.

When the at least one passage limiting element starts to deflect from its starting position the passage cross-section between the pull-out body and the reception is reduced initially. When the second end of the at least one passage limiting element moves further toward the reception another flow path is created between the second end and the pull-out body, which leads through the passage of the pull-out body. Thus, two flow paths for the shear thickening fluid can be provided, wherein each has a smaller passage cross-section than the passage cross-section which is being provided when the at least passage limiting element is in its starting position.

Compared to the viscosity of the shear thickening fluid in the starting position of the at least one passage limiting element in the two flow paths there is a higher viscosity due to the smaller passage cross-section of each flow path.

If the pull-out body has several passage limiting elements they should be disposed in their starting position in a way that they are deflected perpendicular to the pull-out direction in opposite directions towards each other. For example, the passage limiting elements can have differently inclined starting positions which facilitate an opposing deflection. Alternatively, for the case that the passage limiting elements are attached to the pull-out body via joints, the joints can be formed in a way that with respect to two passage limiting elements the joints can only be deflected in opposite directions.

In a further embodiment the at least one passage limiting element may have at least one protrusion which protrudes into the passage cross-section. If the pull-out body is pulled out of the reception under high speed, the shear thickening fluid presses on the at least one protrusion, thus, due to the leverage the at least one protrusion directs the at least passage limiting element out of its starting position. The at least one protrusion is constructed in a way that at low pull-out speeds which are caused by physiological movements, it creates negligible low whirlings in the shear thickening fluid, and a deflection of the at least one passage limiting element from its starting position does not occur.

In a further embodiment the at least one passage limiting element may have a pouch in order to redirect a part of the shear thickening fluid into a passage of the pull-out body, wherein the opening of the pouch points into the movement direction of the pull-out body, preferably to the pull-out direction of the pull-out body.

Thus, at low pull-out speeds, for example, due to physiological body movements, on the one hand the shear thickening fluid can flow through the passage cross-section between the passage limiting element and the reception and on the other hand the shear thickening fluid can flow through the additional passage cross-section of the pouch through the pull-out body. The viscosity of the shear thickening fluid in both the flow paths is defined by the size of the passage cross-section through which the shear thickening fluid can flow. The passage cross-section between the at least one passage limiting element and the reception is limited by an edge of the pouch opening and the inner surface of the reception. The passage cross-section of the pouch is limited by the edge of the pouch opening and the outer surface of the pull-out body.

The passage cross-section between the at least one passage limiting element and the inner surface of the reception and the passage cross-section of the pouch can have different sizes when viewed perpendicular to the pull-out direction. These different passage cross-section dimensions of both the possible fluid paths can lead to a different viscosity behavior of the shear thickening fluid when flowing through the passage cross-section between the at least one passage limiting element and the inner surface of the reception, and through the passage cross-section of the pouch.

If the passage cross-section of the pouch is smaller than the passage cross-section between the at least one passage limiting element and the inner surface of the reception compared to the flow through the passage cross-section between the at least one passage limiting element and the inner surface of the reception, the flow of the shear thickening fluid through the pouch opening can be limited earlier. When the pull-out speed is high due to whirlings which are caused by a back-log of the shear thickening fluid the viscosity of the shear thickening fluid can be increased. Thus, the passage for the shear thickening fluid through the pouch opening can be blocked. Then, the shear thickening fluid can only flow through the passage cross-section between the at least one passage limiting element and the inner surface of the reception. Hence, at large pull-out speeds the passage cross-section can be changed by means of the reduction of the number of possible flow paths for the shear thickening fluid.

In a embodiment between the reception and the pull-out body a membrane may be provided in parallel to the pull-out direction, wherein the membrane has an inlet opening in the area of a second end of the reception for receiving shear thickening fluid and an outlet opening in the area of a first end of the reception for letting out shear thickening fluid, wherein the membrane divides the inside of the reception into a first chamber for receiving the pull-out body and a second chamber, wherein a stripper is disposed in front the inlet opening in order to lead the shear thickening fluid from the first chamber through the inlet opening into the second chamber.

Thus, the shear thickening fluid which is transported into the direction of the second end of the reception due to at least one passage limiting element disposed at the pull-out body, can be caught by the stripper and transported through the inlet opening of the membrane. The membrane extends from the first end of the reception towards the second end of the reception and enclosed a volume with the inner surface of the reception.

The second chamber runs parallel to the pull-out body. Due to the flexible properties of the membrane the membrane can extend to a direction perpendicular to the pull-out direction depending on the amount of shear thickening fluid in the first chamber or the second chamber.

When the pull-out body is pulled out of the reception, the pull-out body can transport shear thickening fluid to the direction of the second end of the reception by means of the at least one passage limiting element. Furthermore, also a wall adhesion of the shear thickening fluid on the pull-out body contributes to the transport of shear thickening fluid in the direction of the second end of the reception. The shear thickening fluid conveyed by the at least one passage limiting element and the pull-out body can be redirected into the inlet opening by means of the stripper upstream of the inlet opening of the membrane. Thereby, the shear thickening fluid is conveyed into the second chamber of the membrane.

The outlet opening may be smaller than the inlet opening, thus, in the second chamber a dynamic pressure is formed, which extends the membrane towards the pull-out body. Thus, in the first chamber a passage cross-section between the at least one passage limiting element and the membrane is reduced.

In a further embodiment the reception may be elastic. Accordingly, when the pull-out body is pulled out of the reception a constriction of the reception perpendicular to the pull-out direction can occur due to the negative pressure in the reception. In particular, the reception areas which run parallel to the pull-out body can contract. Thus, the passage cross-section for the shear thickening fluid between the pull-out body and the reception can be reduced.

In a further embodiment the reception may comprise a weaving structure, in particular a pulling grip, which encloses a reception body. A weaving structure can enclose a volume. Due to a tensile load which is caused by an unphysiological movement of the user, the weaving structure can constrict or compress the volume. This is due to a flux of force between pull-out body and reception, which is based on an increased viscosity of the shear thickening fluid resulting from the unphysiological movement.

The weaving structure of the reception is lengthened in pull-out direction, thus, constricting the weaving structure transverse to the pull-out direction. The inner surface of the reception moves toward the pull-out body and, thus, reduces the passage cross-section between the pull-out body and the reception. Thus, a change of the passage cross-section between the pull-out body and the reception is enabled, which extends over the entire length of the overlapping area of pull-out body and reception.

A reduced passage cross-section between pull-out body and reception leads to an increase of the viscosity of the shear thickening fluid and, thus, to a limitation of the range of motion of the orthopedic device.

In terms of low force, physiological movements where only low forces are introduced into the pull-out body or the weaving structure there is no constriction of the weaving structure. This is because in terms of physiological movements the shear thickening fluid only has a low viscosity and, thus, no flux of force or only a low flux of force is provided between the pull-out body and the reception. The pull-out body and the reception rather slide relatively towards each other due to the low viscosity of the shear thickening fluid, which is based on a large passage cross-section in the starting position of the orthopedic device.

Additionally, between the weaving structure and an outer circumferential surface of the reception spacers can be disposed preventing that a little constriction of the weaving structure already effects the reception. Accordingly, the passage cross-section between the pull-out body and the reception remains constant until the constriction of the weaving structure continues beyond the spacers.

For example, the spacers can be elastic, preferably in the form of spring elements. Thereby, the reduction of the passage cross-section between the pull-out body and the reception can be prevented by means of a contraction of the weaving structure until the exhaustion of the spring deflection.

Moreover, by means of the elastic spacers, for example in the form of spring elements, after the tensile load of the weaving structure is omitted, the weaving structure can be returned to its starting position. The reception is flexible in order to turn the weaving structure to its starting position, in particular, to its circumference in the starting position, after the tensile load of the weaving structure is omitted and in order to restore the passage cross-section between the pull-out body and the reception, which was present in the starting position.

In a further embodiment a pulling grip for closing the reception filled with the shear thickening fluid may be disposed between the reception and the pull-out body, wherein a first end of the pulling grip may be circumferentially attached to an upper section of the pull-out body and a second end of the pulling grip may be circumferentially attached to an opening edge of the reception.

The pulling grip is connected to the pull-out body on a first end in a way that the first end of the pulling grip follows the longitudinal movements of the pull-out body in pull-out direction and opposite the pull-out direction. On a second end the pulling grip is connected to the opening edge of the opening of the reception. Accordingly, the pulling grip sits on the reception like a lid and defines the volume in which the shear thickening fluid is housed with respect to the vicinity.

When the pull-out body is pulled out of the reception, thus, the first end of the pulling grip departing from the second end of the pulling grip, the inner surface of the pulling grip starts to move towards the pull-out body. Hence, a passage cross-section which is formed perpendicular to the pull-out direction between the pull-out body and an inner surface of the pulling grip can be reduced. The more the pull-out body is pulled out of the reception the more the passage cross-section between the pull-out body and the inner surface of the pulling grip is reduced. Thereby, the viscosity of the shear thickening fluid in the area between the pull-out body and the inner surface of the pulling grip increases leading to a limitation of the movement capacity of the orthopedic device.

In a further embodiment the at least one passage limiting element may be a torsion body which is twistable around the longitudinal axis of the at least one passage limiting element from a starting position to a limiting position and from the limiting position to the starting position, wherein in the starting position a narrow side of the at least passage limiting element points into a pull-out direction and in a limiting position a broad side of the at least one passage limiting element points into the pull-out direction.

Thus, the at least one passage limiting element can experience a torsion. The reason for the torsion are the forces caused by the pull-out speed, which acts on the at least one passage limiting element. The twist of the at least one passage limiting element can be caused by the torsional moment created when pulling out the pull-out body having at least one passage limiting element with an asymmetrical geometry with respect to the pull-out direction resulting in an asymmetrical incident flow. Already in the starting position the at least one passage limiting element can have a slightly twist along its longitudinal axis and can be bent around an axis of curvature which extends perpendicular to the longitudinal axis of the at least passage limiting element. However, other options for creating the torsional moment to the at least one passage limiting element can be used such as at least one unilaterally disposed protrusion in the area of the second end of the at least one passage limiting element.

If the at least one passage limiting element is twisted around a rotation angle the broad side of the at least passage limiting element points to the pull-out direction according to the amount of the rotation angle. Thus, on the one hand the projected area with respect to the pull-out direction of the at least one passage limiting element is increased which results in an increased pull-out resistance and an increased formation of turbulences and whirlings. The projected area is created when the at least one passage limiting element is projected parallel to the pull-out direction onto an area perpendicular to the pull-out direction. On the other hand, the distance between the inner surface of the reception and the at least one passage limiting element is reduced which creates a smaller passage cross-section. With the increase of the rotation angle of the at least one passage limiting element the pull-out resistance and due to the decreased passage cross-section also the shear rate and, thus, the viscosity of the shear thickening fluid increase. The rotation angle increases with the increase of the pull-out speed accordingly.

Due to this operating principle and the forces varying in consequence the at least one passage limiting element undergoes a twist of a varying degree depending on the pull-out speed. Due to the configuration of the at least passage limiting element a functional geometry can be achieved by means of which at a physiological movement and a low pull-out speed the at least one passage limiting element cannot twist and the passage cross-section cannot be changed. To the contrary, at an unphysiological movement and a high pull-out speed the at least passage limiting element undergoes a large torsional moment due to the forces caused by the flowing fluid, which leads to a strong twist of the at least one passage limiting element. This twist leads to a narrowing of the passage cross-section and, thus, to an increase of the viscosity.

In a further embodiment the reception may have a balancing membrane at one end, which can balance a change of volume within the reception caused by a movement of the pull-out body. Thus, when pulling out the pull-out body a vacuum effect can be prevented, which can disadvantageously affect the viscosity of the shear thickening fluid and, furthermore, can cause an additional back-holding force to the pull-out body. In other words, the balancing membrane provides a pressure compensation within the reception. Compared to a rigid reception, in particular, in the area of a physiological movement, an increased ease of operation of the orthopedic device can be provided.

In a further embodiment the distance between the pull-out body and the reception perpendicular to the pull-out direction in the starting position may amount between 0.01 to 1000 mm, preferably 0.1 to 50 mm, and more preferably 0.1 to 15 mm and even more preferably 0.1 to 5 mm. On the one hand, the distance can be matched to the properties of the used shear thickening fluid and on the other hand to the application of the orthopedic device.

In a further embodiment the at least one passage limiting element may comprise natural rubber or plastic, preferably silicon or thermoplastic resin, more preferably polypropylene, polyethylene and/or polyurethane. Thus, the required strength and stiffness can be provided for the at least one passage limiting element and, furthermore, also elastic properties of the at least passage limiting element can be realized on the basis of these materials.

In a further embodiment the shear thickening fluid may comprise a dispersion of ethylenglycol or silicone oil and siliciumdioxid, preferably silica gel with a particle size of 2 to 1000 nm, surfaces of 30 to 250 m²/g, and a solid content of 5 to 80 weight-% and stabilizers.

### BRIEF DESCRIPTION OF THE DRAWINGS

Further embodiments and aspects of the present device are explained by means of the following description of the figures in more detail.

FIG. 1 shows a schematic side view of a cross-section through an orthopedic device in a starting position;

FIG. 2 shows a schematic cross-section side view of the orthopedic device in a position differing from the starting position;

FIG. 3a shows a schematic cross-sectional top view of the orthopedic device of FIG. 2;

FIG. 3b shows a schematic cross-sectional top view of the orthopedic device of FIG. 3b;

FIG. 4 shows a schematic cross-sectional detailed view of a pull-out body comprising a recess;

FIG. 5 shows a cross-sectional side view of an orthopedic device with a pull-out body in a starting position comprising a passage;

FIG. 6 schematically shows a cross-sectional side view of the orthopedic device of FIG. 5 in a position differing from the starting position;

FIG. 7 schematically shows a front view of a pull-out body with passages and passage limiting elements disposed therein;

FIG. 8 schematically shows a cross-sectional side view of an orthopedic device with a pull-out body in a starting position according to FIG. 5 with a protrusion on the passage limiting element;

FIG. 9 schematically shows a cross-sectional side view of the orthopedic device according to FIG. 7 with a passage limiting element having a protrusion;

FIG. 10 schematically shows a cross-sectional side view of an orthopedic device in a starting position with an anchor-shaped passage limiting element;

FIG. 11 schematically shows a cross-sectional side view of the orthopedic device according to FIG. 10 in a position differing from the starting position;

FIG. 12a schematically shows a cross-sectional front view of an orthopedic device in a starting position with a passage limiting element in the form of a torsion body;

FIG. 12b schematically shows a cross-sectional top view of the orthopedic device according to FIG. 12a;

FIG. 13a schematically shows a cross-sectional front view of the orthopedic device according to FIG. 12a in a position differing from the starting position;

FIG. 13b schematically shows a cross-sectional top view of the orthopedic device according to FIG. 13a;

FIG. 14 schematically shows a cross-sectional side view of an orthopedic device in the starting position with a passage limiting element in the form of a pouch;

FIG. 15 schematically shows a cross-sectional side view of the orthopedic device according to FIG. 14 in a position differing from the starting position;

FIG. 16 schematically shows a cross-sectional side view of an orthopedic device in a starting position with a membrane;

FIG. 17 schematically shows a cross-sectional side view of the orthopedic device according to FIG. 16 in a position differing from the starting position;

FIG. 18 schematically shows a cross-sectional side view of an orthopedic device in a starting position with a balancing membrane at the end of the reception;

FIG. 19 schematically shows a cross-sectional side view of an orthopedic device with a balancing membrane at the end of the reception in a position differing from the starting position;

FIG. 20a schematically shows a cross-sectional front view of an orthopedic device in a starting position with a passage limiting element in the form of a shield;

FIG. 20b schematically shows a cross-sectional top view of the orthopedic device according to FIG. 20a;

FIG. 21a schematically shows a cross-sectional front view of the orthopedic device according to FIG. 20a in a position differing from the starting position;

FIG. 21b schematically shows a cross-sectional top view of the orthopedic device according to FIG. 21a;

FIG. 22 schematically shows a diagram of the development of the viscosity over the related shear rate with respect to different passage cross-sections, respectively;

FIG. 23 schematically shows an ankle joint orthosis with an orthopedic device; and

FIG. 24 schematically shows a wrist orthosis with an orthopedic device.

### DETAILED DESCRIPTION OF PREFERRED EMBODIMENTS

Hereafter preferred embodiments are described by means of the figures. Thereby, the same elements, similar elements or elements with the same effect are indicated by identical reference numerals. To avoid redundancies the following description partially goes without a repeated description of these elements.

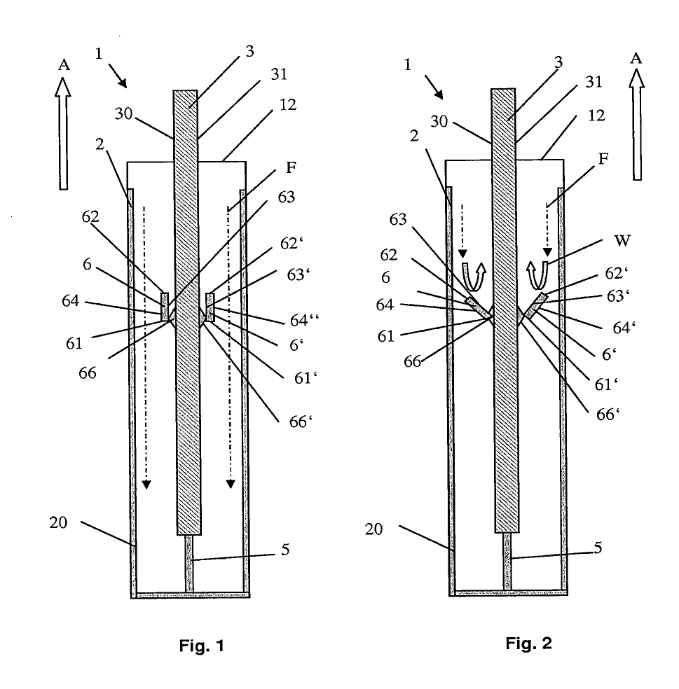

FIG. 1 schematically shows an orthopedic device 1 for limiting the movement of a joint disposed between a first and a second body portion. The device 1 comprises a reception 2 filled with a shear thickening fluid, which can be fixed to a first body portion, and a pull-out body 3, which can be fixed to a second body portion and is movable relatively to the reception 2 in a pull-out direction A and partially extends to the inside of the reception 2 so that between the reception 2 and the pull-out body 3 a passage cross-section filled with the shear thickening fluid is provided.

In the present case, the pull-out body 3 is made of polyamide and the reception 2 is made of polyurethane. Alternatively, both parts can be made of different plastics, such as silicone, rubber, or of a thermoplastic material, such as polyamide, polypropylene, polyethylene or polyurethane. In particular, in terms of the reception 2 it is important that the used material is fluid impermeable.

In the embodiment shown in FIG. 1 the pull-out body 3 has a passage limiting element 6, 6′ on each of a front side 30 and a back side 31. The passage limiting element 6, 6′ has a first surface 63, 3′ which faces the pull-out body surface, and a second surface 64, 64′, which faces the inner surface 20 of the reception 2. Furthermore, the passage limiting element 6, 6′ comprises a first end 61, 61′, which is connected with the pull-out body 3, and a second end 62, 62′, which is free standing and points into the direction of the pull-out direction A. In FIG. 1 the passage limiting element 6, 6′ is connected to the pull-out body 3 on its first end 61, 61′ via a joint 66, 66′, here in the form of a film joint. In this case, the film joint is provided as a local reduction of the wall thickness of the passage limiting element 6, 6′. Thus, the bending stiffness of the passage limiting element 6, 6′ in the area of the film joint is reduced. Thus, the passage limiting element 6, 6′ is disposed in a manner foldable around the joint 66, 66′, in this case the film joint, relative to the pull-out body 3.

In the starting position the passage limiting element 6, 6′ is aligned parallel to the pull-out body 3, hence, in pull-out direction A. The two passage limiting elements 6, 6′ are attached at the same height on the pull-out body 3 with respect to the pull-out direction A. Thus, when the pull-out body 3 is pulled out of the reception 2, on the front side 30 and the back side 31 of the pull-out body 3 symmetrical flow conditions of the shear thickening fluid or a symmetrical fluid flow F relative to the pull-out body 3 and, thus, a symmetrical load distribution within the orthopedic device 1 is created. Alternatively, the passage limiting elements 6, 6′ can also be disposed in an offset manner.

The passage limiting element 6, 6′ in FIG. 1 is configured in a way that even at a deflection of 90 degree to the pull-out direction A it does not contact the inner surface 20 of the reception 2. The bending stresses created in the film joint when the passage limiting element 6, 6′ is deflected, cause the passage limiting element 6, 6′ to return itself to the load-free state. Due to the configuration of the film joint it is not possible for the passage limiting element 6, 6′ to take-up an angle greater than 90 degrees to the pull-out direction A. Thus, a stop at the joint 66, 66′ for limiting the movement of the passage limiting element 6, 6′ can be provided. Also, a means for returning the passage limiting element 6, 6′ or for increasing the reflection resistance around the joint 66, 66′, such as a bending spring, can be provided. In a further alternative the passage limiting element 6, 6′ can be configured in a way that the second end 62, 62′ contacts the inner surface 20 of the reception 2 at a maximum deflection and, thus, defines the maximum angle of the deflection of the passage limiting element 6, 6′.

In an alternative embodiment the passage limiting element 6, 6′ can also be connected directly to the pull-out body 3 without a joint connection. Alternatively, the pull-out body 3 can also include several passage limiting elements 6, 6′ on its front side 30 and its back side 31, which at least in a section of the pull-out body 3 are regularly disposed. The pull-out body 3 has the same number of passage limiting elements 6, 6′ on the front side 30 and the back side 31. In the present case, the passage limiting element 6, 6′ is of the same material as the pull-out body 3. Alternatively, the passage limiting element 6, 6′ can also be of a different material, preferably a different thermoplastic resin and be connected to the pull-out body 3 by means of a multi-component manufacturing process.

In the embodiment of the orthopedic device 1 shown in FIG. 1 the pull-out body 3 is movably held in the reception 2 in a rail guide, not shown. The pull-out body 3 can retract into or extend from the reception 2 in the pull-out direction A. Alternatively the pull-out body 3 can also have spacers in the form of local protrusions in order to guide the pull-out body 3 through the reception 2 in a defined manner.

In the embodiment shown in FIG. 1 the orthopedic device 1 has returning means 5 for the pull-out body 3, which after a movement of the pull-out body 3 out of the starting position into a position differing from the starting position moves the pull-out body 3 back into the starting position. Depending on the physiological conditions of the joint disposed between the first body portion and the second body portion, the returning means 5 can be necessary in order to return the joint after being deflected into a resting position. The returning means 5 is chosen in a way that the joint undergoes a gentle reset or that the pull-out body 3 is moved through the shear thickening fluid so that no substantial rise of the viscosity in the shear thickening fluid is induced. Alternatively, the returning means 5 can also be configured in a way that it is solely provided for the reset of the pull-out body 3 into the starting position and does not influence the position of the joint. In this case the return means 5 enables that the pull-out body 3 which, for example, underwent a change of position in a position differing from the starting position, in particular about an offset in the pull-out direction A, by means of an elongation of the joint, also returns in its starting position after a reset of the joint in its resting position.

The return means 5 shown in FIG. 1 is an elastic plastic in the form of a rubber band with the aforementioned properties. Alternatively, the return means 5 can also be formed by a spring, by a pair of permanent magnets, wherein one permanent magnet is disposed at the lower end of the reception 2 and the other permanent magnet is disposed at the end of the pull-out body 3 facing the lower end of the reception 2, or by an elastic sealing. Furthermore, the reset of the pull-out body 3 can also be caused by a negative pressure present in the reception 2. The negative pressure is created when the pull-out body 3 is pulled out along the pull-out direction A.

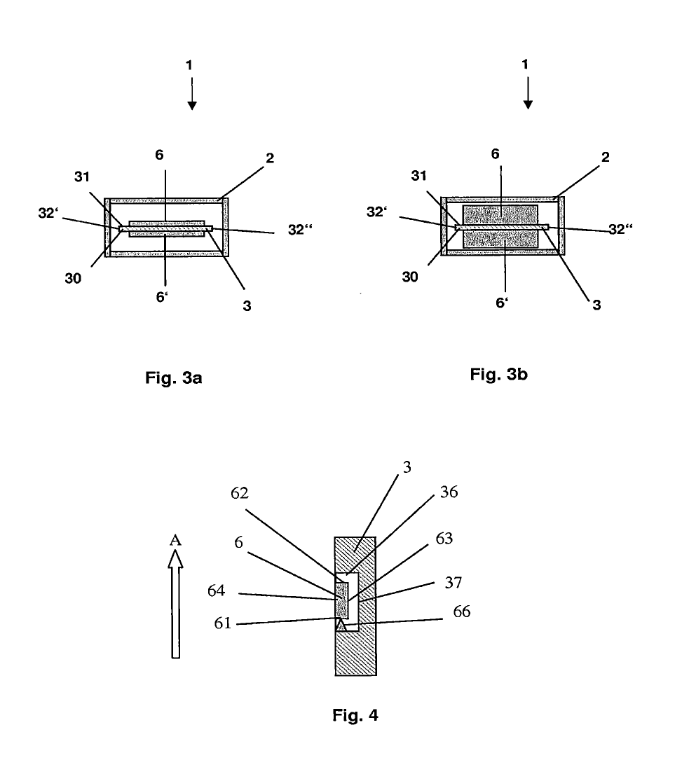

Furthermore, FIG. 1 schematically shows a sealing 12 which is disposed on the upper end of the reception 2, which is open to the outer vicinity, and seals the inside of the reception 2 against the outer vicinity. The sealing 12 runs around the front side 30, the back side 31 and the lateral surfaces 32′ and 32″ (see FIG. 3) of the pull-out body 3. The pull-out body 3 can be moved relatively to the sealing 12. A circumferential sealing lip of the sealing 12 prevents the shear thickening fluid from leaking out of the inside of the reception 2. In the present case the sealing is formed of thermoplastic polyurethane, alternatively, the sealing can be formed of a different plastic such as latex.

Alternatively, the sealing 12 can be formed like a bellows. A first end of the sealing bellows is firmly connected with the reception 2 and a second end of the sealing bellows is firmly connected with the pull-out body 3. When the pull-out body 3 moves out of the reception 2 the distance between the first and the second end of the sealing bellows increases. Accordingly, the sealing bellows follows the movement of the orthopedic device 1 or the pull-out body 3.

Furthermore, a sealing bellows additionally can provide the function of the return means. In this case, the bellows body of the sealing 12 has an elasticity which enables returning to a resting position after a deflection of the orthopedic device 1, wherein the first end and the second end of the sealing bellows approach each other. An elastic rubber is used as sealing material.

When the pull-out body 3 is moved into or out of the reception 2 the movement of the pull-out body 3 is affected by the properties of the shear thickening fluid. A movement of the pull-out body 3 relative to the reception 2 creates an initiation of a shear force into the shear thickening fluid due to the side adhesion between the shear thickening fluid and the pull-out body surfaces 30, 31 or the inner surface 20 of the reception 2. Due to the viscosity of the shear thickening fluid a force in the direction of the pull-out direction A can be transferred from the pull-out body 3 to the reception via the shear of the shear thickening fluid. The amount of the forces to be transferred depends on the speed of the pull-out body 3 relative to the reception 2 and to a high degree on the passage cross-section.

When under a low force impact or with a physiological speed the orthopedic device 1 retracts or extends, due to the low viscosity of the shear thickening fluid only a low shear force generation occurs. Thus, the pull-out body 3 can easily be moved in the shear thickening fluid.

In case of a pull-out movement the shear thickening fluid flows around the passage limiting elements 6, 6′ which are configured in a way that due to the forces caused by the flow acting on the passage limiting elements 6, 6′ at physiological pull-out speeds they do not or only to a little degree move out of their starting position. The position of the passage limiting elements 6, 6′ substantially corresponds to the starting position.

FIG. 2 shows the orthopedic device 1 of FIG. 1 in a position differing from the starting position. With respect to the starting position the passage limiting element 6, 6′ is deflected around the joint 66, 66′ in the direction of the inner surface 20 of the reception 2. Thus, the second end 62, 62′ of the passage limiting element 6, 6′ is closer to the inner wall of the reception compared to the starting position. Thereby, the passage cross-section in the area of the passage limiting element 6, 6′ is reduced compared to the starting position. Accordingly, the shear rate of the shear thickening fluid compared to the shear rate of the shear thickening fluid in the starting position is substantially higher in the area of the passage limiting element 6, 6′, leading to an increase of the viscosity of the shear thickening fluid in these areas when the pull-out body 3 is moved in pull-out direction A, and, thus, the resulting shear rates in the shear thickening fluid are substantially higher compared to the non-deflected passage limiting element 6, 6′.

The deflection of the passage limiting elements 6, 6′ occurs at pull-out speeds above the physiological area due to the flow dynamic pressure forces acting on the passage limiting elements 6, 6′ caused by the flow resistance of the passage limiting elements 6, 6′ in the shear thickening fluid. Due to their geometry the passage limiting elements 6, 6′ are folded around the joints 66, 66′ in the direction of the inner surface 20 of the reception 2.

The higher the forces acting on the orthopedic device 1 or the higher the speeds, which extend or retract the reception 2 and the pull-out body 3 with respect to each other, the higher are the shear rates occurring between the pull-out body 2 and the shear thickening fluid. With the increase of the shear rates also the viscosity of the shear thickening fluid rises. The shear thickening fluid has a discrete jump in dilatancy at which a rapid, significant increase of the viscosity can be registered. In terms of a high viscosity present in the shear thickening fluid, in particular beyond the jump in dilatancy, the reception 2 and the pull-out body 3 of the orthopedic device 1 cannot or only slightly be moved with respect to each other or for a further movement particularly high forces are required.

When the passage limiting elements 6, 6′ are in the deflected position the shear rate of the shear thickening fluid in the area of the passage limiting elements 6, 6′ at unphysiological pull-out speeds is above the critical shear rate at which the jump in dilatancy occurs. This requires a particularly high viscosity of the shear thickening fluid whereby, a high degree of force transfer between the pull-out body 3 and the reception 2 is generated and a high retention force occurs which acts against a pull-out force responsible for the pull-out of the pull-out body 3. The movement of the pull-out body 3 and, thus, the movement of the first and the second body portion between which the orthopedic device 1 is disposed is slowed down or completely impeded.

Compared to the starting position the unfolded passage limiting elements 6, 6′ cause an increase of turbulences and whirlings W in the shear thickening fluid due to the stronger redirection of the shear thickening fluid and the higher flow resistance of the passage limiting elements 6, 6′. This also leads to an increase of the retention force described above.

In an alternative embodiment of the orthopedic device instead on the pull-out body the passage limiting elements can also be provided on the inner surface of the reception. Here, the orientation of the passage limiting elements with respect to the pull-out direction is reversed. In other words, the second end points contrary to the pull-out direction. Alternatively, the passage limiting elements can be provided on one side of the pull-out body, for example the front side, and the second side, here the back side accordingly, can be realized without passage limiting elements. The part of the inner surface of the reception which faces the second side, here the back side accordingly, one or a plurality of passage limiting elements can be provided. A combination of passage limiting elements on the inner surface of the reception and the pull-out body is also possible.

FIG. 3a schematically shows a cross-sectional top view of the orthopedic device 1 of FIG. 1. The passage limiting elements 6, 6′ are present on the front side 30 as well as on the back side 31 of the pull-out body 3 in the starting position and, thus, in a folded state. The front side 30 and the back side 31 of the pull-out body 3 are many times larger than both the lateral surfaces 32′, 32″ of the pull-out body 3. Thus, the front side 30 and the back side 31 and the passage limiting elements 6, 6′ present thereon are responsible for the majority of the shear force transfer from the pull-out body 3 to the shear thickening fluid.

In comparison FIG. 3b schematically shows a cross-sectional top view of the orthopedic device 1 of FIG. 2. The strongly reduced passage cross-section due to the unfolded position of the passage limiting elements 6 can be significantly recognized. Furthermore, due to the unfolded position of the passage limiting elements 6 the pull-out resistance in pull-out direction A is increased due to the increased projected area of the passage limiting elements 6. When the pull-out body 3 is moved into the pull-out direction the shear thickening fluid experiences a stronger redirection. Furthermore, more turbulences and whirlings W are created.

FIG. 4 schematically shows a cross-sectional detailed view of a pull-out body 3 having a recess 36. The first surface 63 of the passage limiting element 6 has a distance to an inner surface 37 of the recess 36 so that during the pull-out movement the shear thickening fluid flows around the first surface 63 of the passage limiting element 6 and, thus, can cause a deflection of the passage limiting element 6 at unphysiological pull-out speeds. In the case of a pull-out body 3 having a plurality of recesses 36 the recesses 36 of the pull-out body 3 with a passage limiting element 6 contained therein can be provided according to the pull-out body 3 of FIGS. 1 and 2, preferably also on the front side 30 and the back side 31 of the pull-out body 3. By means of recessing the passage limiting elements 6 in the recess 36 the total distance of the pull-out body 3 to the reception perpendicular to the pull-out direction A can be used as passage cross-section for the shear thickening fluid. By means of a force impact onto the orthopedic device 1 uncritical for the joint to be preserved, the pull-out body 3 and the reception can be moved relatively towards each other, wherein the passage cross-section is not influenced or reduced by the passage limiting elements 6.

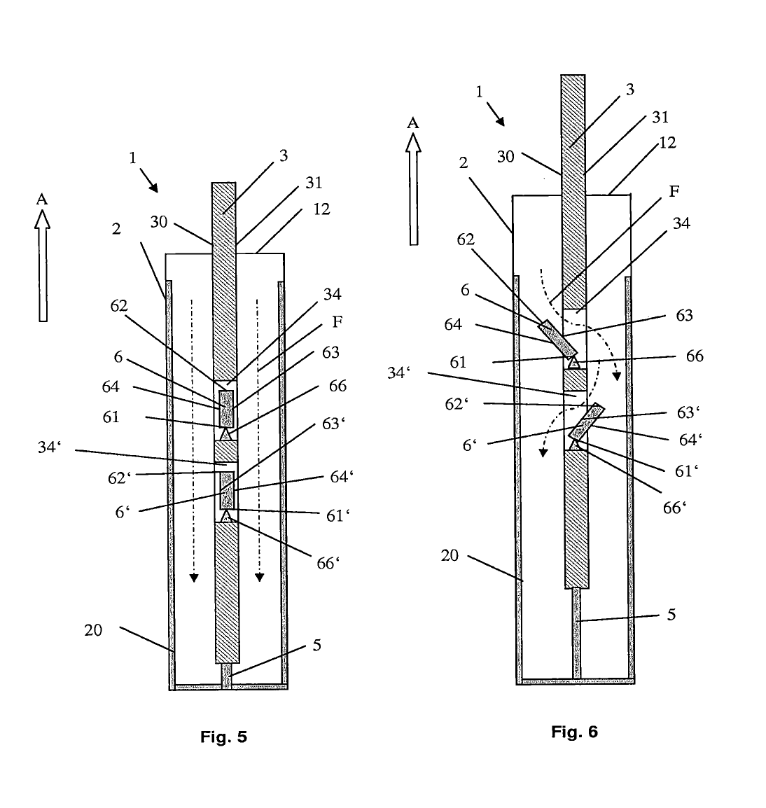

FIG. 5 schematically shows a cross-sectional side view of an alternative embodiment of an orthopedic device in a starting position. The pull-out body 3 has passages 34, 34′, which completely pass through the pull-out body 3 from the front side 30 to the back side 31. The passage limiting elements 6, 6′ are disposed in the passages 34, 34′ of the pull-out body 3 and are connected to the pull-out body 3 by means of joints 66, 66′. The passage limiting elements 6, 6′ are configured in parallel to the pull-out direction A, thus, no local reduction of the passage cross-section occurs as it is the case in the configuration of the passage limiting elements 6, 6′ in FIG. 1. Overall, little component heights of the orthopedic device 1 can be realized. No additional gap is needed between the pull-out body 3 and the reception 2 in order to provide the passage limiting elements 6, 6′. Due to the low flow redirection of the fluid flow F when the pull-out body 3 having passage limiting elements 6, 6′ with this geometry is moved, less viscosity increasing turbulences and whirlings W occur. This enables a high ease of operation of the movement of the pull-out body 3 in pull-out direction A.

In the present embodiment the passage limiting elements 6, 6′ are disposed centrally in the passages 34, 34′. As the wall thickness of the passage limiting elements 6, 6′ is lower than the one of the pull-out body 3, the first surface 63, 63′ or the second surface 64, 64′ of the passage limiting elements 6, 6′ are offsetted to the inside with respect to the front side 30 or the back side 31, respectively. The material slots for realizing the film joints of the passage limiting elements 6, 6′ are disposed alternatingly with respect to the central axis of the pull-out body 3 so that the passage limiting elements 6, 6′ each are deflected in the direction of the front side 30 and the back side 31 of the pull-out body 3, respectively, in terms of unphysiological pull-out speeds. On the one hand an even load distribution on the pull-out body 3 can be achieved. On the other hand the positive effect for reducing the passage cross-section of the fluid achieved by the passage limiting elements 6, 6′ can be used on both sides of the pull-out body 3.

In an alternated embodiment the passage limiting elements 6, 6′ can also align with the surface of the pull-out body 3 which lies in the folding direction of the respective passage limiting element 6, 6′ or can protrude there from. The passage limiting elements 6, 6′ can also have the same or a higher wall thickness than the pull-out body 3 and also be disposed eccentrically in the passage 34, 34′. Furthermore, instead of the connection by means of a joint 66, 66′ also a rigid connection between the passage limiting elements 6, 6′ and the pull-out body 3 can be provided.

FIG. 6 shows the orthopedic device 1 of FIG. 5 in a position differing from the starting position. The passage limiting elements 6, 6′ are deflected in the direction of the inner surface 20 of the reception 2. The passage cross-section between the inner surface 20 and the reception 2 and the corresponding second end 62, 62′ of the passage limiting elements 6, 6′ is reduced compared to the starting position. Due to the deflection of the passage limiting elements 6, 6′ in the passages 34, 34′ of the pull-out body 3 the passages 34, 34′ are open. In terms of a movement of the pull-out body 3 fluid can flow through the passages 34, 34′. Thus, in terms of a movement of the pull-out body 3 the fluid flow F of the shear thickening fluid is divided in a way that a first part of the shear thickening fluid flows through the passage cross-section and a second part of the shear thickening fluid flows through the passage 34, 34′ of the pull-out body 3. Compared to the flow path of the flow through the passage cross-section in the starting position in both the flow paths of the flow of the shear thickening fluid a higher shear rate is present so that the shear thickening fluid has an increased viscosity in both divided flow paths.

In order to facilitate the deflection of the passage limiting elements 6, 6′ the geometry of the passages 34, 34′ and the passage limiting elements 6, 6′ is configured in a way that when the pull-out body 3 is moved in pull-out direction A, a slightly asymmetrical incident flow of the passage limiting elements 6, 6′ occurs. For example, this can be realized by means of radius of different sizes at the corresponding edges of the top side 34a of the passages 34, 34′ as well as opposite on the corresponding edges of the second end 62, 62′ of the passage limiting elements 6, 6′, not shown in FIG. 6. Alternatively, also other geometric designs can be used in order to achieve the asymmetrical incident flow. Furthermore, in the starting position the passage limiting elements 6, 6′ can be arranged slightly inclined with respect to the pull-out direction A under a small angle in the direction of the inner surface 20 of the reception 2.

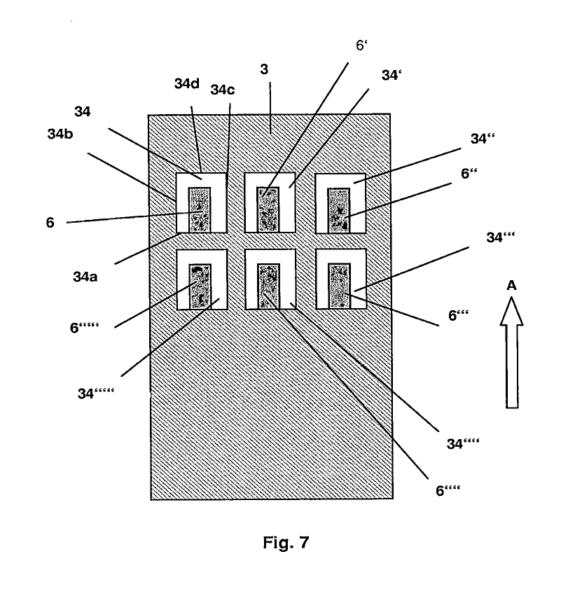

FIG. 7 schematically shows a front view of the pull-out body 3 of FIGS. 5 and 6 with passages 34, 34′, 34″, 34′″, 34″″, 34′″″ and passage limiting elements 6, 6′, 6″, 6′″, 6″″, 6′″″ present therein; the passages 34 to 34′″″ are provided continuously and provided in two rows of three pieces each. The passage limiting elements 6 to 6′″″ present in the passages 34 to 34′″″ each are provided centrally on the bottom side 34a of the passages 34 to 34′″″ and have a defined distance to the lateral edges 34b, 34c, and the top side 34d of the passages 34 to 34′″″.

Due to the opposing deflection of the passage limiting elements 6, 6′ shown in FIG. 6 the forces created by the passage limiting elements 6, 6′, acting on the pull-out body 3, can be balanced to the maximum possible extent. Accordingly, in FIG. 7 with respect to one passage limiting element 6 to 6′″″ the fold-out direction of the adjacent passage limiting elements 6 to 6′″″ in the same row and of the passage limiting elements 6 to 6′″″ in the next row directly above or below opposes to the fold-out direction of the one passage limiting element 6 to 6′″″.

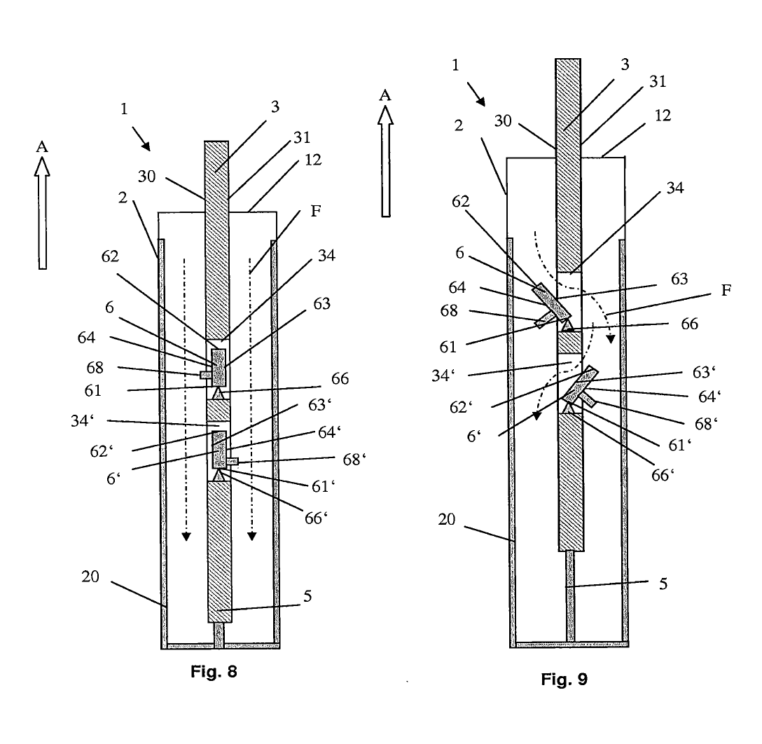

FIG. 8 shows an embodiment of the orthopedic device 1 similar to the one of FIG. 5 in a starting position. The passage limiting elements 6, 6′ have a protrusion 68, 68′ disposed at a first end 61, 61′ on the first surface, which extends perpendicular to the pull-out direction A and protrudes into the passage cross-section without significantly influencing it. In the present embodiment the joint 66, 66′ is a film joint according to FIG. 1 which is configured such that when the pull-out body 3 is pulled out with a physiological pull-out speed due to the caused leverage no or only a very little deflection of the passage limiting element 6, 6′ occurs and the passage cross-section is not or only insignificantly changed in this area. Furthermore, due to the protrusion 68, 68′ only negligibly low turbulences and whirlings W are created in the shear thickening fluid at these speeds.

The orthopedic device 1 of FIG. 8 is shown in FIG. 9 in a position different from the starting position. The passage limiting elements 6, 6′ are deflected towards the inner surface 20 of the reception 2. The orthopedic device 1 has this position in terms of an unphysiological body movement, for example. The deflection of the passage limiting elements 6, 6′ occurs due to the leverage of the flow dynamical forces onto the respective protrusion 68, 68′. The effect of the deflection of the passage limiting elements 6, 6′ on the viscosity of the shear thickening fluid corresponds to the one of the FIGS. 2 and 6. The protrusions 68, 68′ can also serve for limiting the deflection movement of the passage limiting elements 6, 6′.

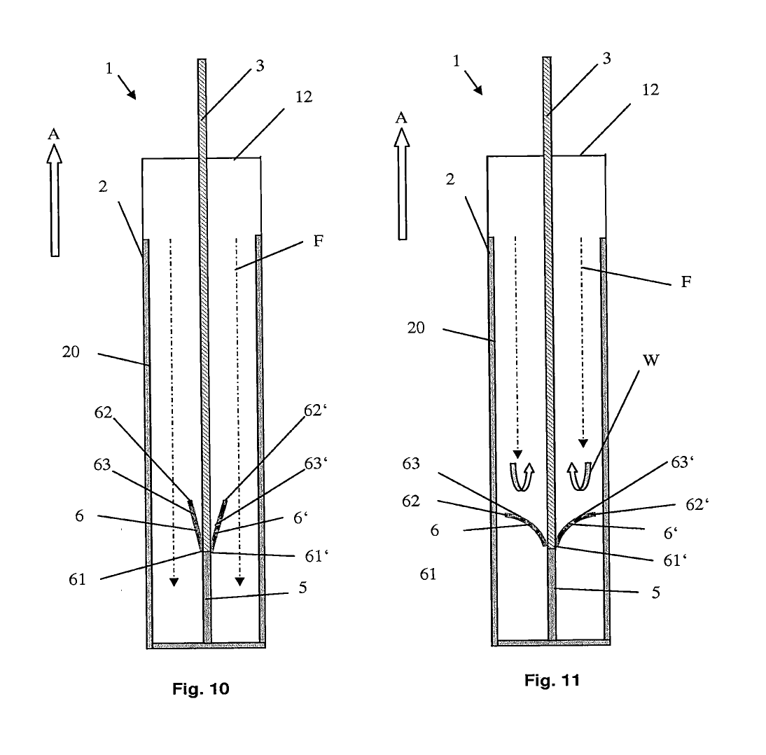

FIG. 10 shows an orthopedic device 1 in a starting position, wherein the pull-out body 3 has an anchor-shaped passage limiting element 6, 6′ each on its front side 30 and on its back side 31. The first ends 61, 61′ of the passage limiting elements 6, 6′ are connected to the pull-out body 3 in a torque-proof manner. In the starting position or the resting position the passage limiting elements 6, 6′ point in pull-out direction A and differ therefrom solely around a small angle in the direction of the inner surface 20 of the reception. Thus, the form corresponds to an anchor with tight flukes. Thereby, the second end 62, 62′ of each passage limiting element 6, 6′ is very close to the pull-out body 3, so that a relatively large passage cross-section is provided. Accordingly, in the areas of the passage limiting elements 6, 6′ the passage cross-section is only insignificantly smaller than the ones between the inner surface 20 of the reception 2 and the smooth front side 30 and back side 31 of the pull-out body.

At physiological body movements the forces caused by the flow only achieve insignificant or no changes with respect to the geometry of the flake-shaped passage limiting element 6, 6′. Accordingly, the geometry of the passage limiting elements 6, 6′ at physiological movements corresponds to the one in the starting position. By means of the configuration of the passage limiting elements 6, 6′ at an acute angle to the pull-out direction A due to the form resulting when the pull-out body 3 is reset from a pulled-out position into the starting position a low flow resistance without or with only little generation of turbulences or whirlings W occurs. Alternatively, any desired number of passage limiting elements 6, 6′ can be attached to the pull-out body 3.

FIG. 11 shows the orthopedic device 1 of FIG. 10 in a position different from the starting position. The passage limiting elements 6, 6′ are bent according to their bending stiffness due to the flow dynamical forces acting on the first surface 63, 63′ in the direction of the inner surface 20 of the reception 2. Thereby, the second end 62, 62′ of each passage limiting element 6, 6′ is closer to the inner surface 20 of the reception 2 reducing the passage cross-section compared to the starting position. This position of the orthopedic device 1 is typically caused by unphysiological body movements.

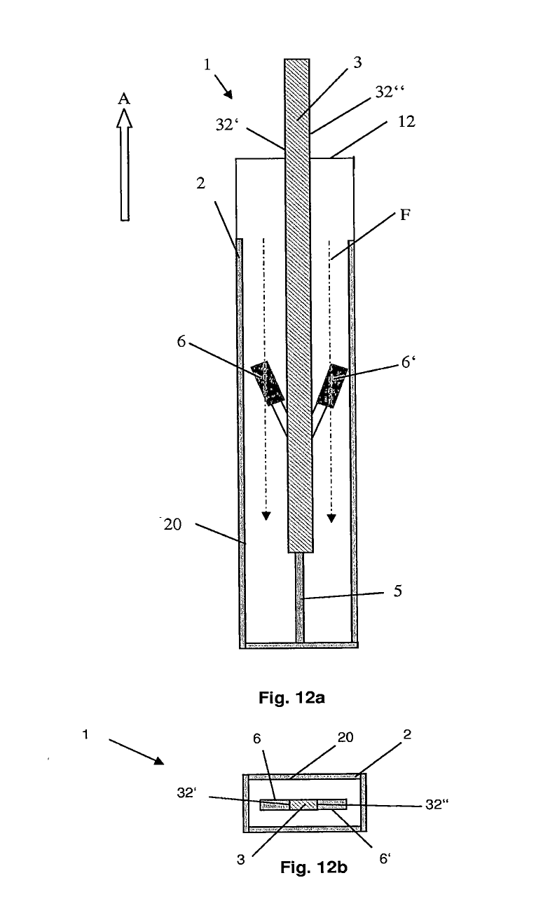

FIGS. 12a and 12b show an embodiment of the orthopedic device 1 with passage limiting elements 6, 6′ in the form of torsion bodies which can be twisted around the longitudinal axis of the passage limiting elements 6, 6′. The passage limiting elements 6, 6′ are disposed on the lateral surfaces 32′, 32″ of the pull-out body 3 and are in a starting position. The passage limiting elements have a narrow side and a broad side. In the starting position the narrow side points in the pull-out direction. Thereby, between the passage limiting elements 6, 6′ and the inner surface 20 of the reception 2 a maximum distance is provided.

In the present case, the twist of the passage limiting elements 6, 6′ arises by means of a torsional moment which when pulling out the pull-out body 3 is caused by an asymmetrical geometry of the passage limiting elements 6, 6′ with respect to the pull-out direction A and an asymmetrical incident flow resulting there from. Already in the starting position, the passage limiting elements 6, 6′ have a slight twist along their longitudinal axis and are bent around an axis of curvature which is perpendicular to the longitudinal axis. Also other options for the generation of the torsional moment onto the passage limiting elements 6, 6′ can be used such as one-sided attached protrusions in the area of the second ends 62, 62′ of the passage limiting elements 6, 6′.

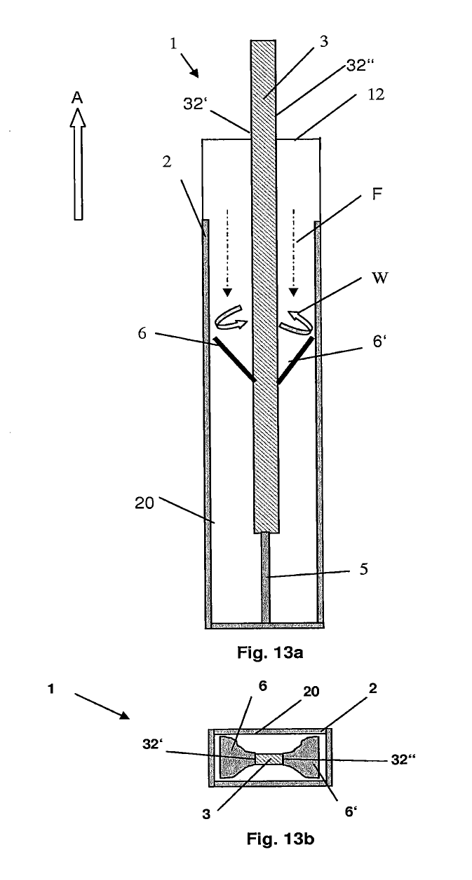

FIGS. 13a and 13b show an orthopedic device 1 similar to the one of FIGS. 12a and 12b in a position differing from the starting position. Compared to the starting position in this position the passage limiting elements 6, 6′ are twisted around a torsional angle so that a majority of the broad side of the passage limiting elements 6, 6′ points in pull-out direction. Thereby, the projected surfaces in pull-out direction A of the passage limiting elements 6, 6′ are increased leading to an increased pull-out resistance and an increased generation of turbulences and whirlings W. The projected areas are generated when the passage limiting elements 6, 6′ are projected parallel to the pull-out direction A onto an area perpendicular to the pull-out direction A. The distance between the inner surface 20 of the reception and the passage limiting elements 6, 6′ is reduced which creates a smaller cross-section. With an increasing torsional angle of the passage limiting elements 6, 6′ the pull-out resistance rises and due to the decreased passage cross-section also the viscosity of the shear thickening fluid rises.

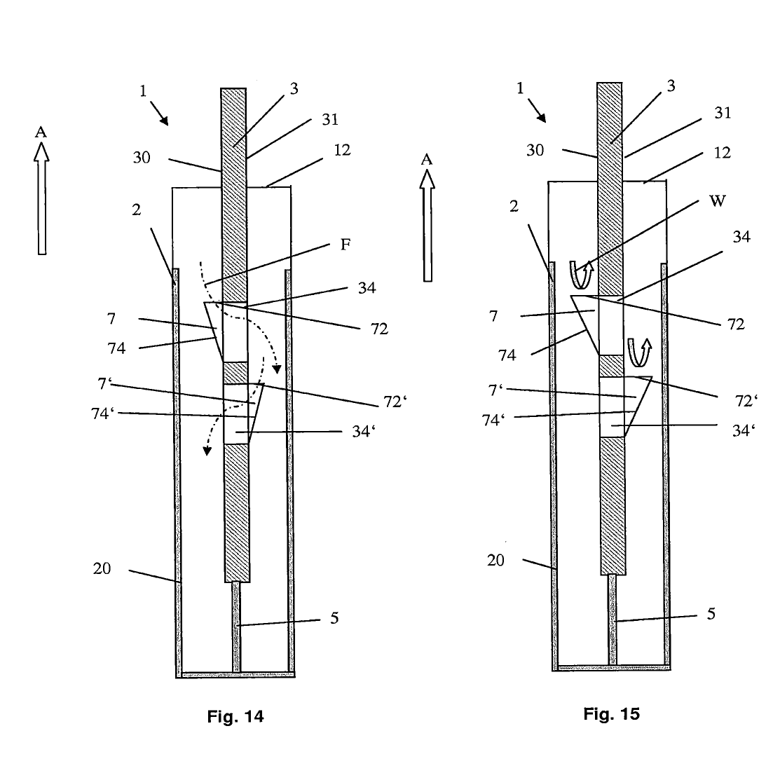

In FIG. 14 two passage limiting elements of an embodiment shown in a starting position of an orthopedic device 1 are configured on the pull-out body 3 in the form of pouches 7, 7′. The pouches 7, 7′ comprise passages 34, 34′ on the pull-out body 3 which have a shell-shaped wall 74, 74′ and a pouch opening 72, 72′ on one side. Upon movement of the pull-out body 3 shear thickening fluid flows through the pouches 7, 7′. Thus, the pouches 7,7′ provide further discrete passage cross-sections. When the pull-out body 3 is pulled out of the reception 2 a first flow path of the shear thickening fluid is created through the passage cross-section between the inner surface 20 of the reception 2 and the pouches 7, 7′, and further flow paths through the pouch openings 72, 72′. The two pouches 7, 7′ are disposed offset from one-another. Alternatively any number of pouches 7, 7′ can be provided on the pull-out body.

FIG. 15 shows the orthopedic device 1 of FIG. 14 in a position differing from the starting position, wherein the device 1 is positioned at an unphysiological body movement. The pouch openings 72, 72′ are blocked due to the high viscosity of the shear thickening fluid, i.e. the shear thickening, in the area of the pouch openings 72, 72′, caused by the high pull-out speeds and the high shear rates resulting there from. The entire flow of the shear thickening fluid is led through the passage cross-section between the inner surface 20 of the reception 2 and the pouch 7, 7′. This results in a strong increase of the shear rate. Additionally, the blocking of the pouch opening 72, 72′ causes an increase of turbulences and whirlings W which also contribute to an increase of viscosity of the shear thickening fluid.

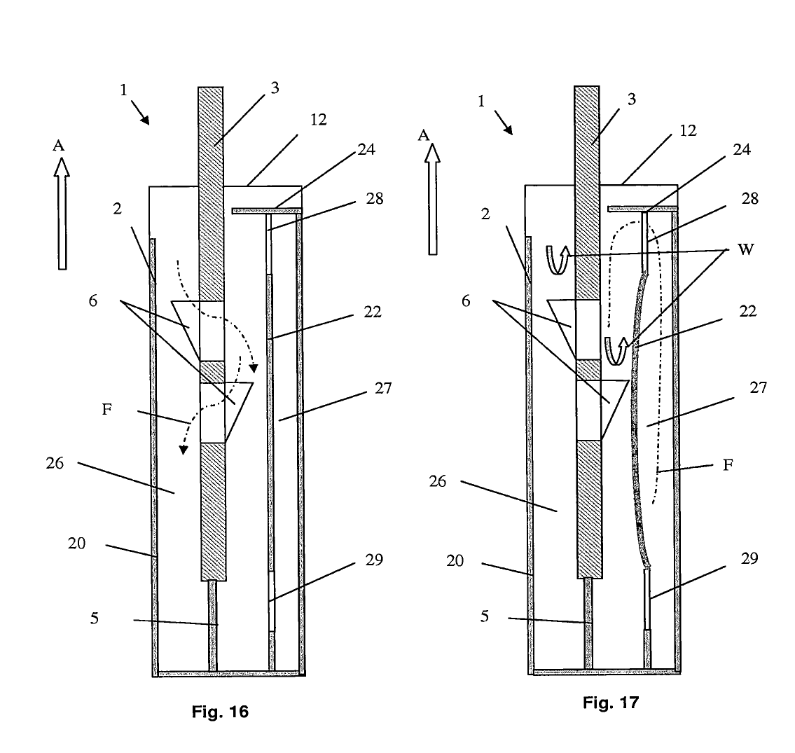

FIG. 16 shows the orthopedic device 1 of FIG. 14 in a starting position, wherein the reception 2 has a membrane 22 which divides the inside of the reception 2 into a first chamber 26 for housing the pull-out body 3 and a second chamber 27. Alternatively, any embodiment of the passage limiting elements 6 of the pull-out body 3 can be combined with the reception 2 having the membrane 22. The membrane 22 has an inlet opening 28 in the area of a second end of the reception 2 for incorporating the shear thickening fluid into the second chamber 27, and in the area of a first end of the reception 2 an outlet opening 29 for letting out the shear thickening fluid from the second chamber 27. Upstream of the inlet port 28 there is a stripper 24 for leading the shear thickening fluid from the first chamber 26 through the inlet opening 28 into the second chamber 27. In this embodiment the inlet port 28 is bigger than the outlet port 29. Alternatively, the proportions can be different depending on the configuration of the orthopedic device 1.

The second chamber 27 runs parallel to the pull-out body 3. Due to the elastic properties of the membrane 22 the latter extends perpendicular to the pull-out direction A towards the pull-out body 3 when the shear thickening fluid flows from the first chamber 26 into the second chamber 27. With the increase of the distance the pull-out body 3 travels in pull-out direction A, the membrane 22 is increasingly pushed in the direction of the pull-out body due to the increase of the shear thickening fluid led into the second chamber 27. This reduces the passage cross-section additionally and provides an even stronger back holding force.

FIG. 17 shows the orthopedic device of FIG. 16 in a position in which the membrane 22 is extended into the direction of the pull-out body 3. In this position the passage cross-section between the passage limiting element 6 and the membrane 22 in the first chamber 26 is additionally reduced by means of the expansion of the membrane 22 perpendicular to the pull-out direction A towards the pull-out body 3.

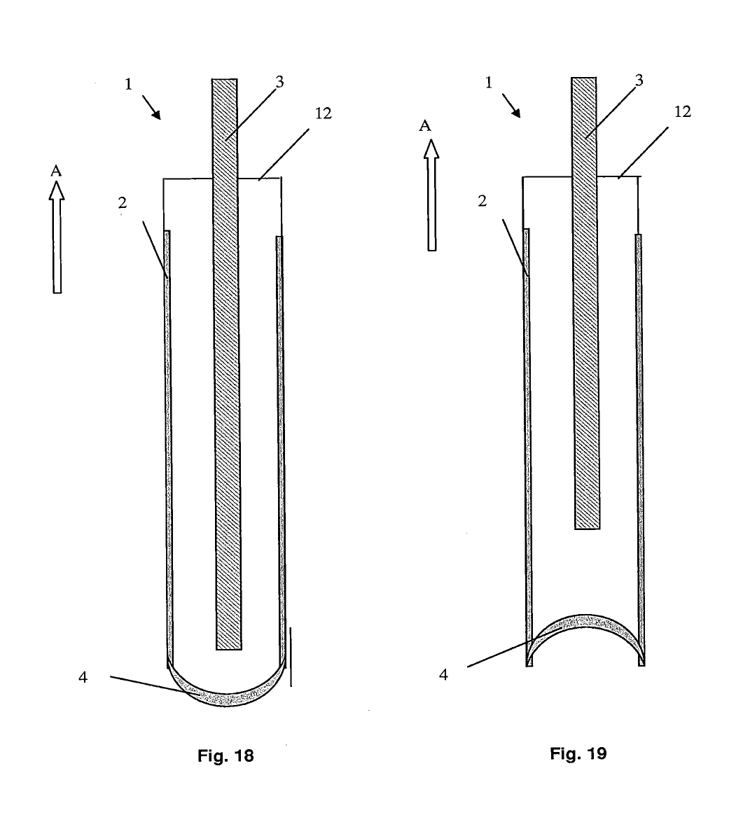

FIG. 18 shows an orthopedic device 1 with a reception 2 having an flexible rubber balancing membrane 4 which is attached to the bottom side of the reception 2. The orthopedic device 1 is in a starting position in which the pull-out body 3 is retracted with respect to the range of motion of the orthopedic device 1. The balancing membrane 4 is of an outwardly curved form with respect to the inside of the reception 2. The entire volume inside the reception 2 is filled with shear thickening fluid up to the sealing 12.

FIG. 19 shows the orthopedic device 1 of FIG. 18 in a position different from the starting position. Compared to the starting position the pull-out body 3 has been pulled out in pull-out direction A about a certain amount, thus, reducing the volume inside the reception 2 accordingly. The balancing membrane 4 balances the created negative pressure by means of bulging into the pull-out direction A according to the replaced volume inside the reception 2. In the present case the balancing membrane 4 has a form curved to the inside with respect to the inside of the reception 2. The balancing membrane 4 can be of any shape that is defined due to the position of the pull-out body 3 relative to the reception 2.

When the pull-out body 3 is pulled out due to the balancing of the volume within the reception 2 a vacuum effect can be prevented which would be disadvantageously for the viscosity of the shear thickening fluid and would provide an additional back holding force to the pull-out body 3. Thus, the balancing membrane 4 provides a pressure compensation within the reception 2, thereby, in particular in the area of physiological movements, a further increased ease of operation of the orthopedic device 1 can be provided compared to a rigid reception 2. Alternatively, the balancing membrane 4 can also be of a different flexible material, such as latex or a thermoplastic polyurethane.

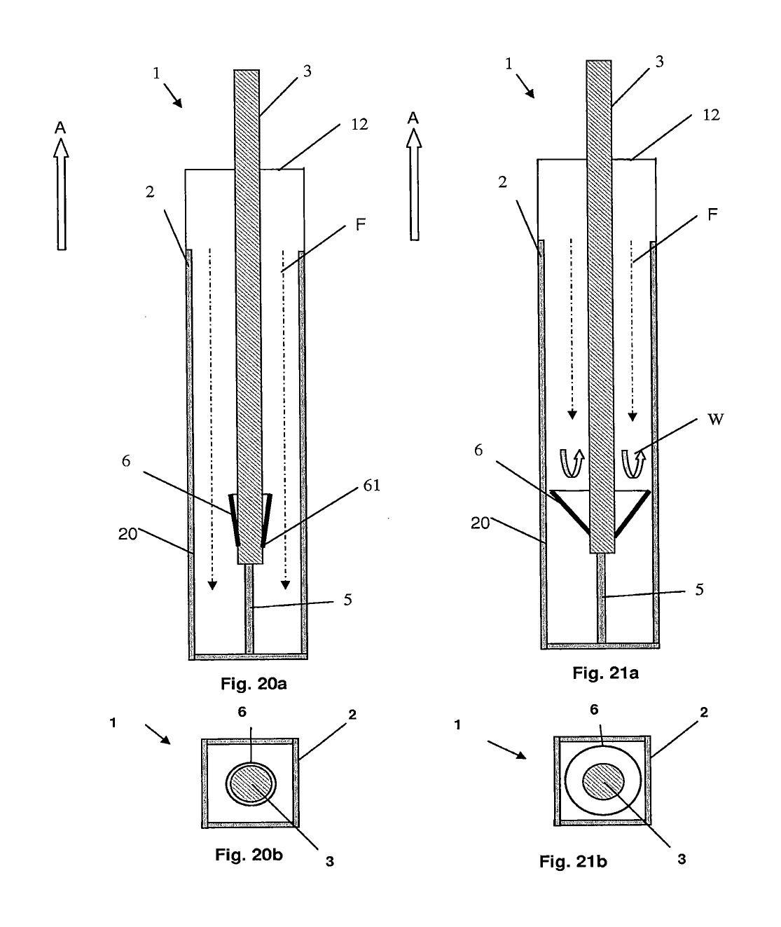

FIG. 20a schematically shows a cross-sectional front view of an orthopedic device 1 with a passage limiting element 6 in the form of a shield in a starting position, such that the pull-out body 3 is surrounded by the passage limiting element 6 like an umbrella. In the starting position the umbrella-like passage limiting element 6 and the pull-out body 3 form a closed shield. The first end 61 of the passage limiting element 6, which surrounds the pull-out body, forms a torque-proof connection with the pull-out body 3. The umbrella-shaped passage limiting element 6 is made of natural rubber.

In terms of physiological pull-out speeds the umbrella-shaped passage limiting element 6 does not undergo any or undergoes only little deflection so that the fluid flow F relative to the pull-out body is only influenced slightly by means of the umbrella-shaped passage limiting element 6.

FIG. 20b schematically shows a cross-sectional top view of the orthopedic device 1 of FIG. 20a. Due to the close fitting of the umbrella-shaped passage limiting element 6 on the pull-out body 3 a larger passage cross-section is provided.

FIG. 21a schematically shows a cross-sectional top view of the orthopedic device according to FIG. 20a in a position differing from the starting position. Compared to the starting position the umbrella-shaped passage limiting element 6 is stretched towards the direction of the reception 2 reducing the passage cross-section between the reception 2 and the umbrella-shaped passage limiting element 6.

FIG. 21b schematically shows a cross-sectional top view of the orthopedic device according to FIG. 21a. Compared to the starting position a reduced passage cross-section is provided by means of the stretched umbrella-shaped passage limiting element 6. The umbrella-shaped passage limiting element 6 is in the stretched position in terms of unphysiological pull-out speeds and provides an increase of the viscosity of the shear thickening fluid between the umbrella-shaped passage limiting element 6 and the reception 2 and an increase of turbulences and whirlings W in the shear thickening fluid.

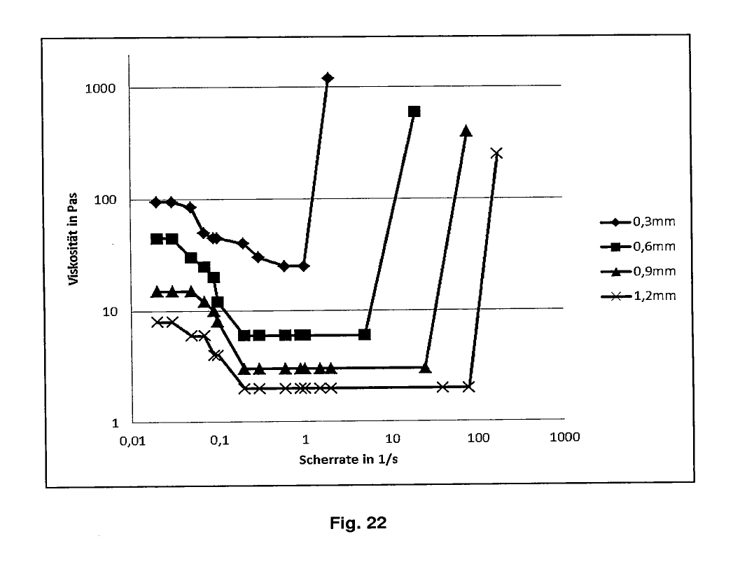

FIG. 22 schematically shows the development of viscosity of a shear thickening fluid, not described any closer herein, with respect to the shear rate for different passage cross-sections, wherein the viscosity is given logarithmically in Pas on the ordinate axis and the shear rate is given in s⁻¹ on the abscissa axis. The passage cross-sections only differ in the distances between the pull-out body and the inner surface of the reception, which are listed in the caption on the right side of FIG. 22. All four graphs show the typical behavior of shear thickening fluids. After an initial shear thinning due to an increasing homogenization of the fluid according to the increased particle movement, at a critical shear rate a rapid increase of the viscosity occurs, namely the jump in dilatancy. In other words, from the jump in dilatancy on there is a sudden shear thickening in the shear thickening fluid.

From FIG. 22 it is evident that with a decreasing passage cross-section on the one hand the viscosity increases at a constant shear rate and on the other hand the jump indilitancy already occurs at a lower shear rate. Thus, by means of a specific constructive adjustment of the passage cross-section of an orthopedic device 1 both the occurring viscosity as well as the position of the jump indilitancy can be adapted to the particular application.

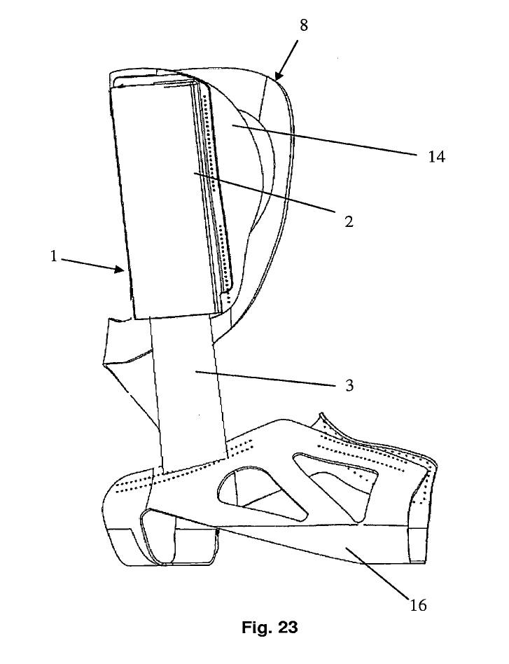

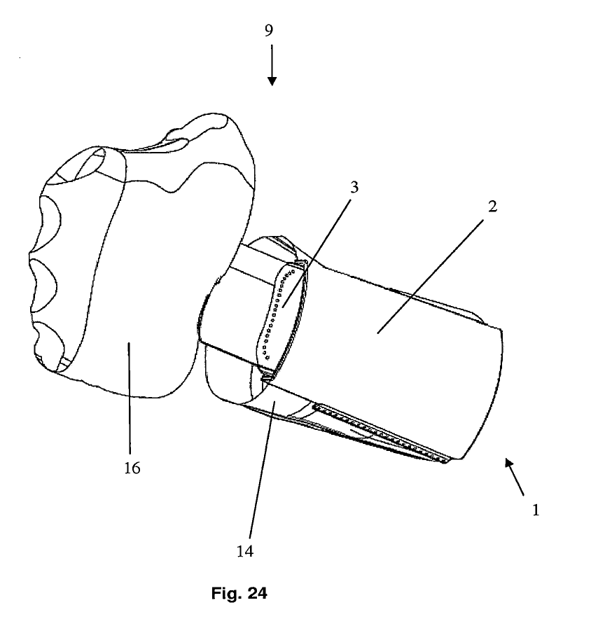

FIG. 23 shows an ankle-joint orthosis 8 which has an orthopedic device 1. The orthosis is disposed around an ankle-joint, not shown, at two body portions, not shown, in a way that the ankle-joint is located between a first orthosis section 14 and a second orthosis section 16. The first orthosis section 14 and the second orthosis section 16 are connected by means of the orthopedic device 1, wherein the reception 2 is attached to the first orthosis section 14 and the pull-out body 3 is attached to the second orthosis section 16. In terms of a supination movement of the ankle-joint the pull-out body 3 is pulled out of the reception 2 in the scale of the radian measure resulting from the angle of the supination and the distance between the pull-out body 3 and the joint center. In the area of physiological movements the pull-out body 3 is not or only slightly restrained. If the supination speed is in the unphysiological area, a movement restriction of the two orthosis sections 14, 16 and, thus, of the two body portions towards each other occurs due to the strong back holding force of the orthopedic device 1 resulting therefrom.

FIG. 24 shows a wrist orthosis 9. The wrist orthosis 9 has a distal first orthosis section 14 and a proximal second orthosis section 16 of a wrist, not shown. The first orthosis section 14 and the second orthosis section 16 are connected by means of the orthopedic device 1 in a way that the pull-out body 3 is attached to the second orthosis section 16 and the reception 2 is attached to the first orthosis section 14. Dorsal extension of the wrist the pull-out body 3 is pulled out of the reception 2 according to the radian measure depending on the dorsal extension angle and the distance between the wrist rotation axis and the device. In terms of physiological movements the pull-out body 3 is not or only slightly restrained. If the dorsal extension speed is in the unphysiological area a restriction of movement of the two orthosis sections 14, 16 and, thus, of the two body portions towards each other occurs due to the resulting strong back holding forces of the orthopedic device 1.

### LIST OF REFERENCE NUMERALS

1 orthopedic device
2 reception
20 inner surface
22 membrane
24 stripper
26 first chamber
27 second chamber
28 inlet opening
29 outlet opening
3 pull-out body
30 front side
31 back side
32′, 32″ lateral surface
34, 34′, 34″, 34′″, 34″″, 34′″″ passage
34a bottom side
34b, 34c lateral edge
34d top side
36 recess
37 inner side
4 balancing membrane
5 reset means
6, 6′, 6″, 6′″, 6″″, 6′″″ passage limiting element
61, 61′ first end
62, 62′ second end
63, 63′ first surface
64, 64′ second surface
66, 66′ joint
68, 68′ protrusion
7, 7′ pouch
71, 71′ pouch opening
74, 74′ shell-shaped wall
8 ankle-joint orthosis
9 wrist orthosis
12 sealing
14 first orthosis section
16 second orthosis section
A pull-out direction
F fluid flow relative to the pull-out body
W turbulences and whirlings

1. An orthopedic device for limiting the movement of a joint disposed between a first body portion and a second body portion, comprising at least one reception fixable to the first body portion, and a pull-out body fixable to the second body portion and movable relative to the reception, wherein perpendicular to a pull out direction between the reception and the pull-out body a passage cross section filled with a shear thickening fluid is provided, and wherein
at least one passage limiting element for changing the passage cross section is provided.

2. The orthopedic device according to claim 1, wherein the at least one passage limiting element is at least one of plate shaped, disc shaped, and rod shaped.

3. The orthopedic device according to claim 1, wherein the at least one passage limiting element is flexible and on a first end firmly, preferably in a torque proof manner, connected to one of the pull-out body and the reception, in order to deflect from a starting position.

4. The orthopedic device according to claim 1, wherein the at least one passage limiting element on a first end is connected to one of the pull-out body and the reception by means of a joint, preferably a hinge, and more preferably a film hinge, in order to deflect from a starting position.

5. The orthopedic device according to claim 1, wherein the at least one passage limiting element is disposed in an acute angle to one of the pull-out body and the reception in a starting position.

6. The orthopedic device according to claim 1, wherein the pull-out body is surrounded by at least one umbrella shaped passage limiting element.

7. The orthopedic device according to claim 1, wherein at least one of the pull-out body and the reception have at least one recess in which at least one passage limiting element is recessed in a starting position.

8. The orthopedic device according to claim 1, wherein the pull-out body has at least one passage for providing an additional flow path for the shear thickening fluid, in which at least one passage limiting element is recessed in a starting position.

9. The orthopedic device according to claim 7, wherein the at least one passage limiting element has at least one protrusion protruding into the passage cross-section for the activation of a deflection of the at least one passage limiting element.

10. The orthopedic device according to claim 1, wherein the at least one passage limiting element has a pouch in order to redirect a part of the shear thickening fluid into a passage of the pull-out body, wherein a pouch opening points in the direction of the movement of the pull-out body, preferably in the pull-out direction of the pull-out body.

11. The orthopedic device according to claim 1, wherein between the reception and the pull-out body a flexible membrane is provided in parallel to the pull-out direction, wherein the membrane has an inlet opening in the area of a second end of the reception for receiving the shear thickening fluid, and an outlet opening in the area of a first end of the reception for letting out the shear thickening fluid, wherein the membrane divides the inside of the reception into a first chamber for receiving the pull-out body and a second chamber, wherein a stripper is disposed in front of the inlet opening in order to lead the shear thickening fluid from the first chamber through the inlet opening into the second chamber.

12. The orthopedic device according to claim 1, wherein the reception is flexible.

13. The orthopedic device according to claim 1, wherein the reception comprises a weaving structure, preferably a pulling grip, which encloses a reception body.

14. The orthopedic device according to claim 1, wherein a pulling grip for closing the reception filled with the shear thickening fluid is disposed between the reception and the pull-out body, wherein a first end of the pulling grip is circumferentially attached to an upper section of the pull-out body, and a second end of the pulling grip is circumferentially attached to an opening edge of the reception.

15. The orthopedic device according to claim 1, wherein the at least one passage limiting element is a torsion body which is twistable around the longitudinal axis of the at least one passage limiting element from a starting position to a limiting position and from the limiting position to the starting position, wherein in the starting position a narrow side of the at least passage limiting element points into a pull-out direction and in a limiting position a broad side of the at least one passage limiting element points into the pull-out direction.

16. The orthopedic device according to claim 1, wherein the reception has an elastic balancing membrane in at least one area, which can balance a change of volume within the reception caused by a movement of the pull-out body.

17. The orthopedic device according to claim 1, wherein the distance between the pull-out body and the reception perpendicular to the pull-out direction in the starting position amounts between 0.01 to 1000 mm, preferably 0.1 to 50 mm, and more preferably 0.1 to 15 mm and even more preferably 0.1 to 5 mm.

18. The orthopedic device according to claim 1, wherein the at least one passage limiting element comprises one of natural rubber and plastic, preferably one of silicon and thermoplastic resin, more preferably at least one of polypropylene, polyethylene and polyurethane.

19. The orthopedic device according to claim 1, wherein the shear thickening fluid comprises a dispersion of one of ethylenglycol and silicone oil and siliciumdioxid, preferably silica gel with a particle size of 2 to 1000 nm, surfaces of 30 to 250 m²/g, and a solid content of 5 to 80 weight-% and stabilizers.
